(19) United States
(12) Patent Application Publication
(10) Pub. No.: US 2009/0110640 A1
(43) Pub. Date: Apr. 30, 2009

(54) CONTRAST AGENTS FOR MAGNETIC RESONANCE IMAGING AND SPECTROSCOPY CONSISTING OF A CYCLIC OLIGOAMID CORE OF 3 TO 4 IDENTICIAL MONOMER UNITS WITH 3 TO 4 PARAMAGNETIC CHELATE SIDE CHAINS

(76) Inventors: Oskar Axelsson, Hagan (NO); Alan Cuthbertson, Oslo (NO); Andreas Meijer, Oslo (NO)

Correspondence Address: GE HEALTHCARE, INC., IP DEPARTMENT, 101 CARNEGIE CENTER, PRINCETON, NJ, 08540-6231, US

(21) Appl. No.: 12/294,263

(22) PCT Filed: Dec. 1, 2006
(86) PCT No.: PCT/NO2006/000448

§ 371 Date: Sep. 24, 2008

(30) Foreign Application Priority Data
Mar. 29, 2006 (NO) 20061434
Aug. 10, 2006 (NO) 20063633

(51) Int. Cl. A61K 49/06; C08G 69/02; A61K 49/00

(52) U.S. Cl. 424/9.3; 528/310; 424/9.1

(57) ABSTRACT

The present invention relates to: Compounds of formula (I) consisting of a cyclic polymer core A and groups -L-X attached to said core A-(L-X)ₙ (I) wherein A denotes a cyclic polymer which is comprised of 3 or 4 identical monomers which are connected by amide bonds; L may be present or not and if present is that same or different and denotes a linker moiety, X is the same or different and denotes a chelator; and n denotes an integer of 3 or 4; Compound of formula (II) consisting of a cyclic polymer core A and groups -L-X′ attached to said core A-(L-X′)ₙ (H) wherein A denotes a cyclic polymer which is comprised of 3 or 4 identical monomers which are connected by amide bonds; L may be present or not and if present is that same or different and denotes a linker moiety, X is the same or different and denotes a paramagnetic chelate consisting of a chelator X and a paramagnetic metal ion M; and n denotes an integer of 3 or 4; And compositions comprising compounds of formula (II) and their use as contrast agents in magnetic resonance (MR) imaging (MRI) and magnetic resonance spectroscopy (MRS).

### COMPOUNDS

The present invention relates to novel compounds of formula (I) and (II), compositions comprising compounds of formula (II) and their use as contrast agents in magnetic resonance (MR) imaging (MRI) and magnetic resonance spectroscopy (MRS).

MR image signal is influenced by a number of parameters that can be divided into two general categories: inherent tissue parameters and user-selectable imaging parameters. Inherent tissue parameters that affect MR signal intensity of a particular tissue are mainly the proton density, i.e. hydrogen nuclei density of that tissue and its inherent T₁ and T₂ relaxation times. Signal intensity is also influenced by other factors such as flow. The contrast between two adjacent tissues, e.g. a tumour and normal tissue depends on the difference in signal between the two tissues. This difference can be maximised by proper use of user-selectable parameters. User-selectable parameters that can affect MR image contrast include choice of pulse sequences, flip angles, echo time, repetition time and use of contrast agents.

Contrast agents are often used in MRI in order to improve the image contrast. Contrast agents work by effecting the T₁, T₂ and/or T₂* relaxation times and thereby influencing the contrast in the images. Information related to perfusion, permeability and cellular density as well as other physiological parameters can be obtained by observing the dynamic behaviour of a contrast agent.

Several types of contrast agents have been used in MRI. Water-soluble paramagnetic metal chelates, for instance gadolinium chelates like Omniscan™ (GE Healthcare) are widely used MR contrast agents. Because of their low molecular weight they rapidly distribute into the extracellular space (i.e. the blood and the interstitium) when administered into the vasculature. They are also cleared relatively rapidly from the body.

Blood pool MR contrast agents on the other hand, for instance superparamagnetic iron oxide particles, are retained within the vasculature for a prolonged time. They have proven to be extremely useful to enhance contrast in the liver but also to detect capillary permeability abnormalities, e.g. “leaky” capillary walls in tumours which are a result of tumour angiogenesis.

The existent paramagnetic metal chelates that are used as MR contrast agents have a low relaxivity at the 1.5 T magnetic field that is standard in most of today's MR scanner. In 3 T systems which probably will dominate or at least be a substantial fraction of the market in the future, the intrinsic contrast is lower, all T₁ values are higher and the hardware will be faster, so the need for a contrast agent with good performance at 3 T is considerable. In general, the longitudinal relaxivity (r1) of contrast agents falls off at the high magnetic fields of the modern MR scanners, i.e. 1.5 T, 3 T or even higher. This is due to the fast rotational Brownian motion of small molecules in solution which leads to weaker magnetic field coupling of the paramagnetic metal ion to the water molecules than anticipated.

Many attempts have been made to produce contrast agents with high relaxivity by incorporating the paramagnetic metal chelates into larger molecules, such as various polymers.

WO-A2-2005/019247 discloses cyclic peptides which may be conjugated to MR imaging agents.

WO-A2-2003/014157 discloses conjugates of peptides and metal complexes which are used as MRI contrast agents.

WO-A2-2002/094873 discloses cyclic peptides which are linked to a paramagnetic chelate.

All these attempts have been of limited success because of fast internal rotations or segmental motions. Another approach are paramagnetic metal chelates that are bound to or do bind to proteins. However such compounds suffer from pharmacological and pharmacokinetic disadvantages like long excretion time or the risk for interactions with protein bound drugs. Further the leakage through normal endothelium into the interstitium is still substantial.

The present invention provides novel compounds that perform well as MR contrast agents at high magnetic fields, i.e. magnetic fields above 1.5 T. The novel compounds are of rigid structure comprising slowly rotating bonds and in addition showing high water exchange rates.

Thus in a first aspect the present invention provides compounds of formula (I) consisting of a cyclic polymer core A and groups -L-X attached to said core

A-(L-X)n (I)

wherein
A denotes a cyclic polymer which is comprised of 3 or 4 identical monomers which are connected by amide bonds;
L may be present or not and if present is that same or different and denotes a linker moiety,
X is the same or different and denotes a chelator; and
n denotes an integer of 3 or 4

The term “chelator” denotes a chemical entity that binds (complexes) a metal ion to form a chelate. If the metal ion is a paramagnetic metal ion, the chemical entity, i.e. complex, formed by said paramagnetic metal ion and said chelator is denoted a “paramagnetic chelate”.

A preferred embodiment of a compound of formula (I) is a compound of formula (II) consisting of a cyclic polymer core A and groups -L-X′ attached to said core

A-(L-X′)n (II)

wherein
A denotes a cyclic polymer which is comprised of 3 or 4 identical monomers which are connected by amide bonds;
L may be present or not and if present is that same or different and denotes a linker moiety,
X′ is the same or different and denotes a paramagnetic chelate consisting of a chelator X and a paramagnetic metal ion M; and
n denotes an integer of 3 or 4

In said preferred embodiment, said paramagnetic chelate consists of the chelator X and a paramagnetic metal ion M, said chelator X and paramagnetic metal ion M form a complex which is denoted a paramagnetic chelate.

Compounds of formula (I) and (II) are rigid compounds which is due to the fact that they contain a rigid cyclic polymer core A. Further, the L-X/L-X′ pendant groups of formula (I) and (II) exert a rotation restriction on the covalent bond between the core and L and/or L and X/X′, if L is present and/or the covalent bond between the core and X/X′, if L is not present such that these bonds rotate preferably less than 10⁷ times/second at 37° C.

In a preferred embodiment, A is comprised of 3 or 4 identical monomers which are polymerized/cyclized by head to tail linkages resulting in an amide bond between the each of the monomers.

In another preferred embodiment, A is comprised of 3 or 4 identical monomers and each of said monomers comprises a 1,2,3-triazole unit, i.e. a unit of formula (IIIa)

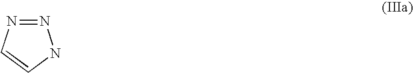

In a preferred further embodiment A is a cyclic polymer of formula (IV)

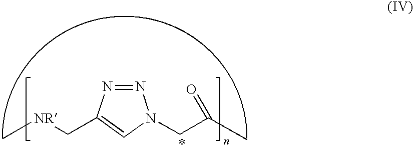

wherein
R′ denotes a group to improve solubility;
* denotes the attachment of the A to L-X or L-X′
n is defined as for formulae (I) and (II) and is preferably 4

R′ is a group that improves solubility of A, e.g. a lower alkyl group, preferably a C₁-C₃-alkyl group which optionally contains heteroatoms like O and N, for instance in the form of hydroxyl groups, ether groups, amino groups, carboxyl groups, ester groups or amide groups or a carboxyl group, an ester group or an amino group.

R′ is preferably selected from the group consisting of H, C₁-C₃-alkyl like CH₃, C₁-C₃-hydroxyalkyl optionally containing an ether group like CH₂OH, OCH₂CH₂OH, C₁-C₃-oxyalkyl like OCH₃, OCH₂CH₃, C₁-C₃-alkoxy like CH₂OCH₃, COOH or C₁-C₃-alkyl esters thereof like COOCH₃ and COOCH₂CH₃, C(O)NH₂ or C₁-C₃-alkylamides like C(O)N(CH₃)₂, C(O)N(CH₂CH₃)CH₃ and C(O)N(CH₂CH₃)₂. Preferred R′ are C₁-C₃-hydroxyalkyl optionally containing an ether group like CH₂OH, OCH₂CH₂OH.

The cyclic polymer A of formula (IV) is cyclized through amide bonds including head-to-tail linkages between the 3 or 4 monomers. The cyclic polymer A is preferably unaffected by enzymatic influence and should not comprise moieties recognisable by enzymes such as hydrolases and peptidases.

A preferred embodiment of compounds of formula (I) and (II), respectively are compounds of formula (Ia) and (IIa)

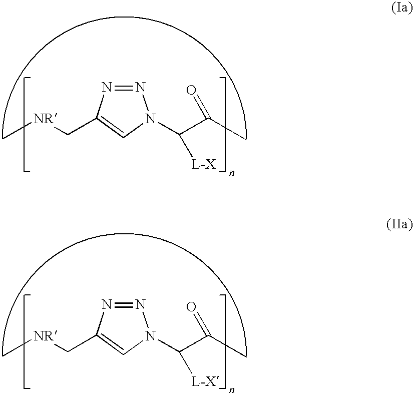

wherein
R′, L, X, X′ and n are as defined above with n being preferably 4.

In another preferred embodiment, A is a cyclic polymer of formula (V)

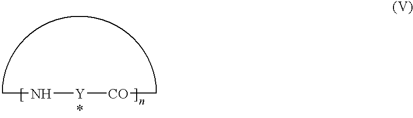

wherein
n is as defined above and preferably 3;
Y denotes a moiety CR1R2-CO-heterocycle or CR1R2-heterocycle, wherein both R1 and R2 are present and are the same or different and denote R′ or only R1 or R2 is present and denotes R′;
* denotes the attachment of the A to L-X or L-X′

Y denotes a moiety CR1R2-CO-heterocycle or CR1R2-heterocycle, wherein R1 and R2 may both be present and are the same or different and denote R′ as defined above, i.e. R1 and R2 are groups that improve the solubility of the cyclic polymer A of formula (V). An example of R1 and R2 being present and R1 being the same as R2 and denote R′ is R1 and R2 being H. An example of R1 and R2 being present and R1 being different from R2 and denote R′ is R1 being H and R2 being CH₂OH.

In another embodiment, only R1 or R2 is present and denotes R′ as defined above, i.e. a group that improves the solubility of the cyclic polymer A of formula (V). In said embodiment, the “free valence” on the C-atom which due to the absence of either R1 or R2 serves as the attachment point of L as defined in formulae (I) and (II).

The heterocycle of Y is preferably selected from oxazole, thiazole, proline or imidazole or derivatives thereof, e.g. derivatives that include groups R′ that improve the solubility of the cyclic polymer A of formula (V). The heterocycle of Y may also serve as the attachment point of L as defined in formulae (I) and (II).

A preferred embodiment of compounds of formula (I) and (II), respectively are compounds of formula (Ib) and (IIb)

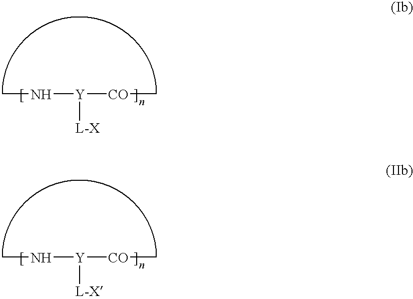

wherein
Y, L, X, X′ and n are as defined above with n being preferably 3.

A preferred embodiment of formula (V) is a cyclic polymer A of formula (VI)

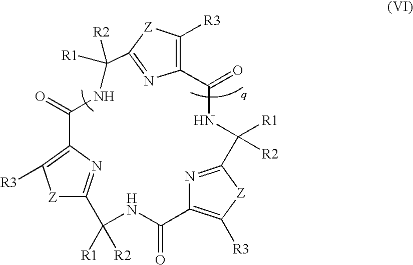

wherein
z denotes O, S or NR4;
R3 denotes R′;
R1 and R2 are defined as for formula (V) above; and
q is an integer of 1 or 2

One of R1, R2, R3 or—if z denotes NR4—R4 is absent and the free valence on the C- or N-atom which is the result of said absence serves as the attachment point of L as defined in formulae (I) and (II). The remaining R1 to R4 denote R′ as defined above, i.e. groups that improve the solubility of the cyclic polymer A of formula (VI).

If z denotes NR4, R4 is preferably absent and the free valence on the N-atom serves as the attachment point of L as defined in formula (I) and (II). In this embodiment, R3 is preferably selected from H and CH₃.

Another preferred embodiment of formula (V) is a cyclic polymer A of formula (VII)

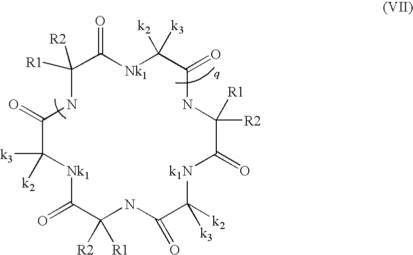

wherein
R1, R2 and q are as defined in formula (VI) above; and
k₁ denotes H or CH₃ and k₁ and either of k₂ or k₃ form a saturated or non-saturated nitrogen heterocycle, preferably a 5- or 6-membered nitrogen heterocycle and most preferably pyrrolidine.

One of R1, R2 and k2/k3 is absent and the free valence on the C-atom which is the result of said absence serves as the attachment point of L as defined in formulae (I) and (II). The remaining R1, R2 or k2/k3 denote R′ as defined above, i.e. groups that improve the solubility of the cyclic polymer A of formula (VII).

Preferably, k₁ and k₂ form pyrrolidone, R1 is absent and the free valence on the C-atom which is the result of R1 being absent serves as the attachment point of L as defined in formulae (I) and (II) and R2 and k₃ denote R′, preferably H.

In compounds of formula (I), formula (II) or preferred embodiments of these compounds, L may be present or not. If L is present, each L is the same or different and denotes a linker moiety, i.e. a moiety that is able to link the core A and X or the core A and X′, respectively. If L is not present, the core A is directly attached to X (compounds of formula (I)) or X′ (compounds of formula (II)) via a covalent bond.

Preferred examples of L are:

Linker moieties —(CZ¹Z²)ₘ-
wherein
m is an integer of 1 to 6; and
Z¹ and Z² independently of each other denote a hydrogen atom, a hydroxyl group or a C₁-C₈-alkyl group optionally substituted by hydroxyl, amino or mercapto groups, e.g. CH₂OH and CH₂CH₂NH₂ and/or optionally comprising an oxo-group, e.g. CH₂OCH₃ and OCH₂CH₂OH.
   Linker moieties —CO—N(Z³)-*
   wherein
* denotes the attachment of the core A to said linker moiety; and
Z³ stands for H, C₁-C₈-alkyl, optionally substituted with one or more hydroxyl or amino groups.
   Linker moieties —CZ¹Z²-CO—N(Z³)-* which are preferred linker moieties,
   wherein
* denotes the attachment of the core A to said linker moiety;
Z¹ and Z² have the meaning mentioned above; and
Z³ stands for H, C₁-C₈-alkyl, optionally substituted with one or more hydroxyl or amino groups.

In a preferred embodiment, Z¹ and Z² are hydrogen or Z¹ is hydrogen and Z² is methyl and Z³ is H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

Linker moieties which are amino acid residues —CZ¹Z²-CO—NH—CH(Z⁴)CO—NH—*

wherein
* denotes the attachment of the core A to said linker moiety;
Z¹ and Z² have the meaning mentioned above, preferably Z¹ and Z² are hydrogen or Z¹ is hydrogen and Z² is methyl; and
Z⁴ stands for the side group of the naturally occurring α-amino acids.
   Linker moieties —CO—NH—CZ¹Z²-*
   wherein
* denotes the attachment of the core A to said linker moiety; and
Z¹ and Z² have the meaning mentioned above, preferably Z¹ and Z² are hydrogen or Z¹ is hydrogen and Z² is methyl

Further preferred examples of L comprise benzene or N-heterocycles such as imidazoles, triazoles, pyrazinones, pyrimidines, piperidines and the core A is attached to either one of the nitrogen atoms in said N-heterocycles or to a carbon atom in said N-heterocycles or in benzene.

Examples of such preferred Ls, wherein * denotes the attachment of the core A to said linker moiety and Q is the same or different and denotes H or methyl, are the following:

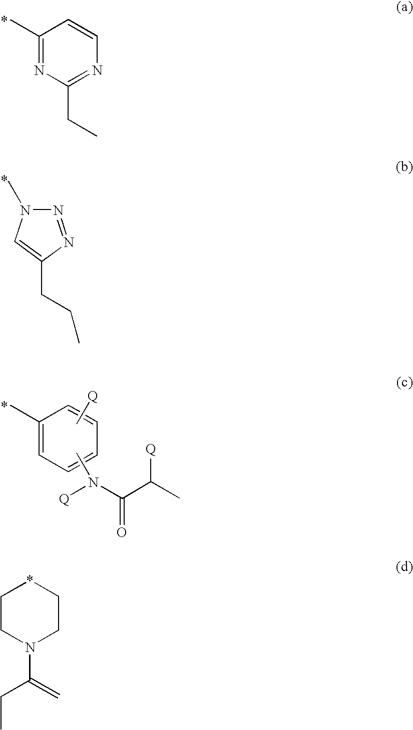

with (d) being a more preferred linker moiety.

Thus a preferred embodiment of compounds of formula (I) and (II), respectively are compounds of formula (Ic) and (IIc)

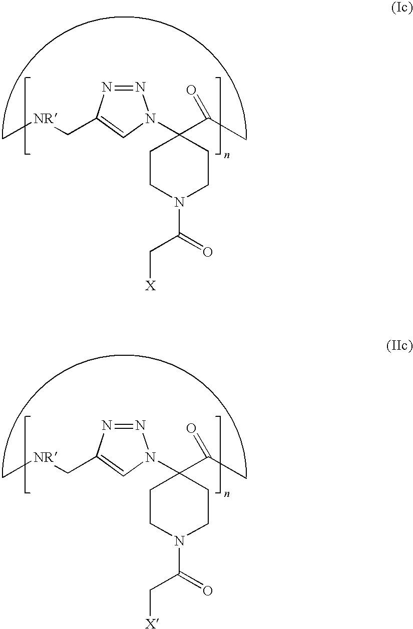

wherein R′, X, X′ and n are defined as above

Preferably, if L is present, all L are the same.

In compounds of formula (I) and preferred embodiments thereof, X is the same or different and denotes a chelator. Preferably, all X are the same.

In compounds of formula (II)—a preferred embodiment of compounds of formula (I)—and preferred embodiments thereof, X is X′ which stands for a paramagnetic chelate, i.e. a chelator X which forms a complex with a paramagnetic metal ion M. In compounds of formula (II) and preferred embodiments thereof, X′ is the same or different. Preferably, all X′ are the same.

Numerous chelators X which form complexes with paramagnetic metal ions M are known in the art. Preferably, X is a cyclic chelator of formula (VIII):

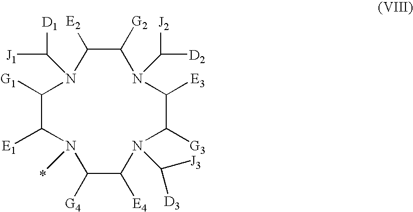

wherein
* denotes the attachment of L, if present, or the core A, if L is not present;
E₁ to E₄ independent of each other is selected from H, CH₂, CH₃, OCH₃, CH₂OH, CH₂OCH₃, OCH₂CH₃, OCH₂CH₂OH, COOH, COOCH₃, COOCH₂CH₃, C(O)NH₂, C(O)N(CH₃)₂, C(O)N(CH₂CH₃)CH₃ or C(O)N(CH₂CH₃)₂;
G₁ to G₄ independent of each other is selected from H, CH₂, CH₃, OCH₃, CH₂OH, CH₂OCH₃, OCH₂CH₃, OCH₂CH₂OH, COOH, COOCH₃, COOCH₂CH₃, C(O)NH₂, C(O)N(CH₃)₂, C(O)N(CH₂CH₃)CH₃, or C(O)N(CH₂CH₃)₂;
D₁ to D₃ independent of each other is selected from H, OH, CH₃, CH₂CH₃, CH₂OH, CH₂OCH₃, OCH₂CH₃, OCH₂CH₂OH or OCH₂C₆H₅; and
J₁ to J₃ independent of each other is selected from COOH, P(O)(OH)₂, P(O)(OH)CH₃, P(O)(OH)CH₂CH₃, P(O)(OH)(CH₂)₃CH₃, P(O)(OH)Ph, P(O)(OH)CH₂Ph, P(O)(OH)OCH₂CH₃, CH(OH)CH₃, CH(OH)CH₂OH, C(O)NH₂, C(O)NHCH₃, C(O)NH(CH₂)₂CH₃, OH or H.

Preferred chelators X are residues of diethylenetriaminopentaacetic acid (DTPA), N-[2-[bis(carboxymethyl)amino]-3-(4-ethoxyphenyl)propyl]-N-[2-[bis(carboxymethyl)-amino]ethyl]-L-glycine (EOB-DTPA), N,N-bis[2-[bis(carboxymethyl)amino]-ethyl]-L-glutamic acid (DTPA-Glu), N,N-bis[2-[bis(carboxymethyl)amino]-ethyl]-L-lysine (DTPA-Lys), mono- or bis-amide derivatives of DTPA such as N,N-bis[2-[carboxymethyl[(methylcarbamoyl)methyl]amino]-ethyl]glycine (DTPA-BMA), 4-carboxy-5,8,11-tris(carboxymethyl)-1-phenyl-2oxa-5,8,11-triazamidecan-13-oic acid (BOPTA), DTPA BOPTA, 1,4,7,10-tetraazacyclododecan-1,4,7-triactetic acid (DO3A), 1,4,7,10-tetraazacyclododecan-1,4,7,10-tetraactetic acid (DOTA), ethylenediaminotetraacetic acid (EDTA), 10-(2-hydroxypropyl)-1,4,7,10-tetraazacyclododecan-1,4,7-triacetic acid (HPDO3A), 2-methyl-1,4,7,10-tetraazacyclododecan-1,4,7,10-tetraacetic acid (MCTA), tetramethyl-1,4,7,10-tetraazacyclododecan-1,4,7,10-tetraacetic acid (DOTMA), 3,6,9,15-tetraazabicyclo[9.3.1]pentadeca-1(15), 11,13-triene-3,6,9-triacetic acid (PCTA), PCTA12, cyclo-PCTA12, N,N′Bis(2-aminoethyl)-1,2-ethanediamine (TETA), 1,4,7,10-tetraazacyclotridecane-N,N′,N″,N′″-tetraacetic acid (TRITA), 1,12-dicarbonyl, 15-(4-isothiocyanatobenzyl) 1,4,7,10,13-pentaazacyclohexadecane-N,N′,N″ triaceticacid (HETA), 1,4,7,10-tetraazacyclododecane-N,N′,N″,N′″-tetraacetic acid mono-(N-hydroxysuccinimidyl) ester (DOTA-NHS), N,N′-Bis(2-aminoethyl)-1,2-ethanediamine-N-hydroxy-succinimide ester (TETA-NHS), [(2S,5S,8S,11S)-4,7,10-tris-carboxymethyl-2,5,8,11-tetramethyl-1,4,7,10-tetraazacyclododecan-1-yl]acetic acid (M4DOTA), [(2S,5S,8S,11S)-4,7-bis-carboxymethyl-2,5,8,11-tetramethyl-1,4,7,10-tetraazacyclo-dodecan-1-yl]acetic acid, (M4DO3A), (R)-2-[(2S,5S,8S,11S)-4,7,10-tris-((R)-1-carboxyethyl)-2,5,8,11-tetramethyl-1,4,7,10-tetraazacyclododecan-1-yl]propionic acid (M4DOTMA), 1 O-Phosphonomethyl-1,4,7, 1-O-tetraazacyclododecane-1,4,7-triacetic acid (MPDO3A), hydroxybenzyl-ethylenediamine-diacetic acid (HBED) and N,N′-ethylenebis-[2-(o-hydroxyphenolic)glycine] (EHPG).

The term “residues of . . . ” in the previous paragraph is chosen since the chelator is attached to the remainder of the molecule represented by compounds of formula (I), (II) and preferred embodiments thereof. Thus, X is to be seen as a residue. The attachment point of X to said remainder of the molecule represented by compounds of formula (I), (II) and preferred embodiments thereof may be any suitable point, e.g. a functional group like a COOH group in a chelator like DTPA, EDTA or DOTA or an amino group in a chelators like DTPA-Lys, but also a non-functional group like a methylene group in a chelators like DOTA.

Suitable chelators X and their synthesis are described in e.g. EP-A-071564, EP-A-448191, WO-A-02/48119, U.S. Pat. No. 6,399,043, WO-A-01/51095, EP-A-203962, EP-A-292689, EP-A-425571, EP-A-230893, EP-A-405704, EP-A-290047, U.S. Pat. No. 6,123,920, US-A-2002/0090342, U.S. Pat. No. 6,403,055, WO-A-02/40060, U.S. Pat. No. 6,458,337, U.S. Pat. No. 6,264,914, U.S. Pat. No. 6,221,334, WO-A-95/31444, U.S. Pat. No. 5,573,752, U.S. Pat. No. 5,358,704 and US-A-2002/0127181, the content of which are incorporated herein by reference.

In a more preferred embodiment of the present invention X is a residue selected from DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

In a particularly preferred embodiment X is a residue selected from DTPA, DOTA, BOPTA, DO3A, HPDO3A, DOTMA, PCTA, DTPA BMA, M4DOTA or M4DO3A.

As stated above, in a preferred embodiment of X, i.e. X′, the chelator X forms a complex, i.e. paramagnetic chelate, with a paramagnetic metal ion M. Suitably, M is selected from ions of transition and lanthanide metals, i.e. metals of atomic numbers 21 to 29, 42, 43, 44 or 57 to 71. More preferred, M is a paramagnetic ion of Mn, Fe, Co, Ni, Eu, Gd, Dy, Tm and Yb, particularly preferred a paramagnetic ion of Mn, Fe, Eu, Gd and Dy. Most preferably M is selected from Gd³⁺, Mn²⁺, Fe³⁺, Dy³⁺ and Eu³⁺ with Gd³⁺ being the most preferred paramagnetic ion M.

Especially preferred compounds are compounds of formula (Ia) and (IIa)

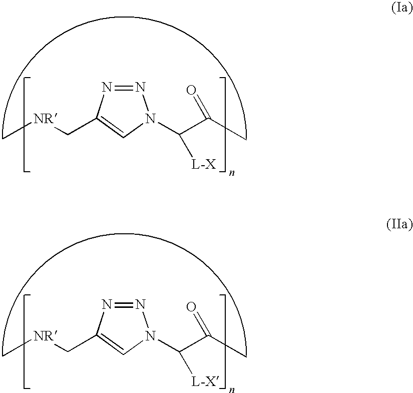

wherein
each L is the same and denotes —CO—N(Z³)-*, wherein * denotes the attachment of the core A to said linker moiety; and Z³ stands for H, C₁-C₈-alkyl, optionally substituted with one or more hydroxyl or amino groups, preferably for H;
each X in formula (Ia) is the same and is selected from the group consisting of residues of DOTA, DTPA, BOPTA, DO3A, HPDO3A, MCTA, DOTMA, DTPA BMA, M4DOTA, PCTA, TETA, TRITA, HETA, DPDP, EDTA and EDTP. More preferably, X is selected from the group consisting of residues of DTPA, DOTA, BOPTA, DO3A, HPDO3A, DOTMA, PCTA, DTPA BMA and M4DOTA;
each X′ in formula (IIa) is the same and the chelator X is as defined in the previous paragraph and the metal ion M is selected from the group consisting of paramagnetic metal ions of Mn, Fe, Eu, Gd and Dy, preferably, the metal ion M is Gd³⁺;
n is as defined previously, preferably 4; and
R′ is H or methyl.

Other especially preferred compounds are compounds of formula (Id) and (IId)

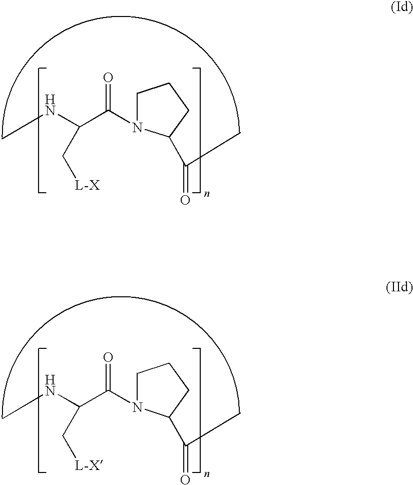

wherein
each L is the same and denotes —CO—N(Z³)-*, wherein * denotes the attachment of the core A to said linker moiety; and Z³ stands for H, C₁-C₈-alkyl, optionally substituted with one or more hydroxyl or amino groups, preferably for H;
each X in formula (Id) is the same and is selected from the group consisting of residues of DOTA, DTPA, BOPTA, DO3A, HPDO3A, MCTA, DOTMA, DTPA BMA, M4DOTA, PCTA, TETA, TRITA, HETA, DPDP, EDTA and EDTP. More preferably, X is selected from the group consisting of residues of DTPA, DOTA, BOPTA, DO3A, HPDO3A, DOTMA, PCTA, DTPA BMA and M4DOTA;
each X′ in formula (IId) is the same and the chelator X is as defined in the previous paragraph and the metal ion M is selected from the group consisting of paramagnetic metal ions of Mn, Fe, Eu, Gd and Dy, preferably, the metal ion M is Gd³⁺;
n is as defined previously, preferably 3.

Other especially preferred compounds are compounds of formula (Ie) and (IIe)

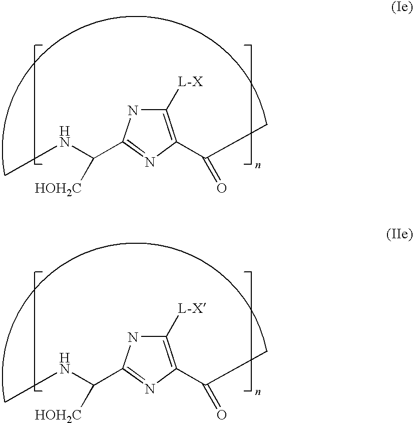

wherein
each L is the same and denotes —CZ¹Z²-CO—N(Z³)-*, wherein * denotes the attachment of the core A to said linker moiety, Z¹ and Z² independently of each other denote a hydrogen atom, a hydroxyl group or a C₁-C₈-alkyl group optionally substituted by hydroxyl, amino or mercapto groups, e.g. CH₂OH and CH₂CH₂NH₂ and/or optionally comprising an oxo-group, e.g. CH₂OCH₃ and OCH₂CH₂OH and Z³ stands for H, C₁-C₈-alkyl, optionally substituted with one or more hydroxyl or amino groups. Preferably, Z¹, Z² and Z³ are H;
each X in formula (Ie) is the same and is selected from the group consisting of residues of DOTA, DTPA, BOPTA, DO3A, HPDO3A, MCTA, DOTMA, DTPA BMA, M4DOTA, PCTA, TETA, TRITA, HETA, DPDP, EDTA and EDTP. More preferably, X is selected from the group consisting of residues of DTPA, DOTA, BOPTA, DO3A, HPDO3A, DOTMA, PCTA, DTPA BMA and M4DOTA;
each X′ in formula (IIe) is the same and the chelator X is as defined in the previous paragraph and the metal ion M is selected from the group consisting of paramagnetic metal ions of Mn, Fe, Eu, Gd and Dy, preferably, the metal ion M is Gd³⁺;
n is as defined previously, preferably 3.

When modelling or mimicking the behaviour of compounds of formula (I) or (II) with theoretical methods and computational techniques (molecular modelling), in a preferred embodiment these compounds can be inscribed in a sphere with a diameter of from 2 to 3.5 nm and preferably in a sphere with a diameter of from 2 to 2.5 nm when using a molecular modelling software that is based on MM3 force field theoretical methods (e.g. the Spartan software) and the compounds are modelled in vacuum.

The compounds of formula (I) and (II) as well as preferred embodiments thereof can be synthesized by several synthetic pathways known to the skilled artisan.

The cyclic polymer core A is comprised of 3 or 4 identical monomers which are connected by amide bonds. The cyclic polymer core A can be synthesized by cyclic polymerization of said monomers by head to tail linkages known in the art, e.g. form peptide chemistry, resulting in an amide bond between the each of the monomers.

Preferably, A is synthesized using the solid-phase methodology of Merrifield employing an automated peptide synthesizer (J. Am. Chem. Soc., 85: 2149 (1964)). Synthesis of peptides (i.e. polymerization of amino acids resulting in an amide bond between the monomers) by solid phase techniques is based upon the sequential addition of protected amino acids linked, optionally through a linker group, to a solid phase support. In one commonly employed method, the α-amino group is suitably protected with acid labile or base labile protecting groups. Following addition and coupling of the first amino acid residue, the α-amino protecting group is removed. The chain is extended by the sequential addition of further protected amino acid derivatives or peptide fragments. After deprotection of relevant amino protecting group the peptide may be cyclized in dilute solution by activating the carboxylic acid functionality.

For the synthesis of the cyclic polymer core A of formula (V), a suitable monomer H₂N—Y—COOH has to be prepared which then can be polymerized and cyclised as described in the previous paragraph.

As for the definition of Y in formula (V), the suitable monomer is either H₂N—CR1R2-heterocycle-COOH (1) or H₂N—CR1R2-CO-heterocycle-COOH (2).

The synthesis of compounds H₂N—CR1R2-heterocycle-COOH, i.e. monomers (1) and the polymerization/cyclization is known in the art, e.g. disclosed in D. Mink et al., Tetrahedron Lett. 1998, 39, 5709-5712. The monomers (1) may be polymerized to trimers or tetramers and cyclised in either a one-pot reaction or in a stepwise manner.

Compounds H₂N—CR1R2-CO-heterocycle-COOH, i.e. monomers (2) may be synthesized by a condensation reaction of H₂N—CR1R2-COOH with an amino acid (proteogenic or non-proteogenic amino acids, D or L form) or a substituted amino acid, i.e. an amino acid wherein the hydrogen atom at the α-C-atom is substituted by other groups, e.g. straight chain or branched alkyl groups, alkenyl groups or alkinyl groups, aryl groups or alkylaryl groups which optionally may contain functional groups like hydroxyl groups and/or heteroatoms like S or O.

In the preferred embodiment of compounds (Id) and (IId), the core A is comprised of monomers (2a) which can be synthesized by a condensation reaction of the amino acid proline and 2,3diaminopropionic acid.

In monomers (1) and (2) R1 and R2 are as defined earlier, i.e. R1 and R2 denote groups that improves solubility of A, e.g. a lower alkyl group, preferably a C₁-C₃-alkyl group which optionally contains heteroatoms like O and N, for instance in the form of hydroxyl groups, ether groups, amino groups, carboxyl groups, ester groups or amide groups or a carboxyl group, an ester group or an amino group.

In another embodiment, either R1 or R2 denote a reactive group which allows the attachment of a linker moiety L. Reactive groups are groups that comprise a reactive moiety, e.g. an activated acid functionality like an acid chloride or amino groups which allow the coupling of an L group or a group L-X/L-X′ by means of e.g. an amide or an ester functionality. Many other attachments can also be considered such as the formation of C—C bonds or heterocyclic groups. It is well known in the science of medicinal chemistry how to use bioisosteric groups to create linkers with similar properties.

Generally, when L is present in the compounds of formula (I), (II) and preferred embodiments thereof, the cyclic polymer core A is preferably prepared as A-(L-T)n, wherein L has a terminal reactive group such as an acid or amine group to react with A or a monomer thereof and T is a leaving group, e.g. chloride when the reactive group is an acid residue. X or X′ is then coupled to the A-(L-)n through a replacement reaction of the leaving group T. A-(L-T)n may be prepared by synthesizing monomers m-(L-T), polymerizing said monomers to a trimer or tetramer (n=3 or 4) and cyclising said trimer or tetramer. Alternatively, monomers are polymerized to obtain a trimer or tetramer (the cyclic polymer core A) and attaching n groups L-T to said core A.

Alternatively, the cyclic polymer core A is prepared in such a way that either R1 or R2 in the monomer denote a reactive group which allows the attachment of L-X or L-X′. Again reactive groups are for instance an activated acid functionality, e.g. an acid chloride or amine groups which allow the attachment of L-XL-X′ by means of e.g. an amide or an ester functionality. Many other attachments can also be considered such as the formation of C—C bonds.

When L is not present in the compounds of formula (I), (II) and preferred embodiments thereof, the cyclic polymer core A is prepared in such a way that either R1 or R2 in the monomer denote a reactive group which allows the attachment of X or X′. Again reactive groups are for instance an activated acid functionality, e.g. an acid chloride or amine groups which allow the attachment of X or X′ by means of e.g. an amide or an ester functionality. Many other attachments can also be considered such as the formation of C—C bonds.

Thus, another aspect of the invention is a process for the preparation of compounds according to formula (Ib), (IIb) and preferred embodiments thereof by
(i) polymerization and cyclization of monomers H₂N—CR1R2-heterocycle-COOH or H₂N—CR1R2-CO-heterocycle-COOH, wherein R1 and R2 are as defined earlier;
(ii) reacting the cyclic polymer core A obtained in step (i) with groups L-X or X, wherein L and X are as defined in claim 1; and
(iii) if compounds of formula (IIb) and preferred embodiments thereof are produced, reacting the reaction product of step (ii) with a paramagnetic metal ion, preferably in the form of its salt.

In another preferred embodiment, if A is a compound of formula (IV), A is obtained by polymerisation of the monomer (3)

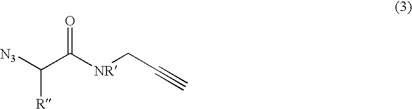

wherein R′ is as defined earlier, i.e. a group improving solubility and R″ is either a group L-T or denotes a reactive group or a precursor thereof which allows the attachment of L, L-X or L-X′, if L is present, or X or X′, if L is not present. As mentioned earlier, a reactive group is a group that comprises a reactive moiety. As an example —CH2-CH2-NH₂ is a reactive group since it comprises a reactive moiety, i.e.—NH₂. A precursor of a reactive group does not comprise a reactive moiety, but a moiety that can be turned into a reactive moiety. An example of a precursor of a reactive group is —CH2-CH2-NO₂ since it does not comprise a reactive moiety, however, by reducing the nitro group to an amino group, a reactive group —CH2-CH2-NH₂ is obtained which comprises the reactive moiety —NH₂.

Monomers (3) may be prepared by a cycloaddition and the cycloaddition of an azide and an alkyne to give 1,2,3 triazole is for instance described in and such cycloadditions are for instance described in Vsevolod et al., Angew. Chem. Int. Ed. 2002, Vol. 41, No. 14, 2596-2599. More preferably, the cycloaddition is copper-catalysed, resulting in 1,4-disubstituted 1,2,3-triazoles. A copper salt, such as CuSO₄, is preferably used, preferably together with a reducing agent such as ascorbic acid and/or sodium ascorbate.

Three or four monomers (3) are polymerized and cyclised, preferably in a one-pot reaction, to prepare A. Computational studies have shown that trimeric and tetrameric structures are preferably generated in such preparation. Further, any unspecific polymerization can be hampered by performing the cyclization in a diluted medium.

If A is a compound of formula (IV) it can be prepared as follows and R′ and R″ are as earlier defined:

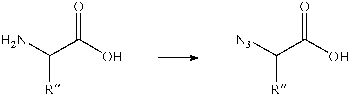

The initial reaction of preparing an azide from an amino acid may be carried out as described by Lundquist et al., Org. Lett. 2001, Vol. 3, No. 5, 781-783.

As shown above, one of the starting materials comprised by the monomers (3) is an amino acid. Relevant amino acids are e.g. selected from lysine, ornithine, 2,3-diaminopropionic acid (Dap), diaminobutyric acid (Dab), amino-glycine (Agl), 4-amino-piperidine-4-carboxylic acid (Pip), allo-threonine and 4-amino-phenylalanine. The functional groups in said amino acids can be used to attach a linker moiety L. The starting materials, i.e. amino acid and alkyne, are commercially available or may be prepared according to methods well known in the art.

The cycloaddition of the azide of the previous step and an alkyne is shown below and results in compounds of formula (IV)

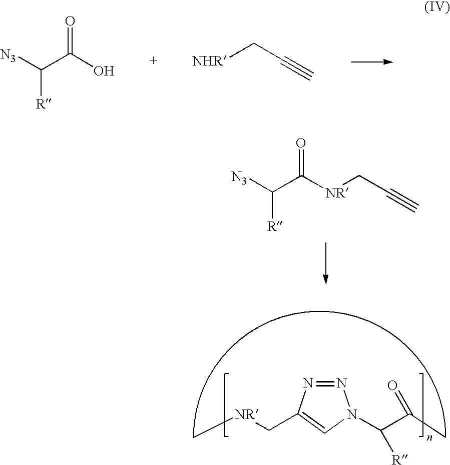

Cyclic polymer cores A of formula (IV) comprising a linker moiety L that comprises a cyclic moiety, i.e. a linker moiety L that comprises benzene or N-heterocycles or any of the linker moieties (a) to (d) may be prepared as described above using amino acids as follows:

Aromatic unnatural amino acids, forming a basis for linker moieties comprising an aromatic structure like benzene can be synthesized by the Strecker synthesis according to A. Strecker. Ann. Chem. Pharm. 75 (1850), p. 27, shown below:

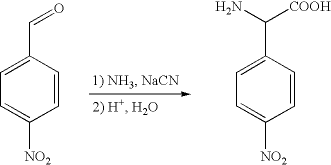

The nitro group is a masked amino functionality (precursor of the reactive moiety —NH₂) that can be generated after cyclization to provide an attachment for X or X′.

4-amino-piperidine-carboxylic acid (Pip) can be synthesised in a similar way, as shown below:

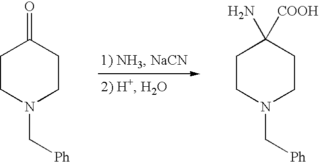

In a preferred embodiment, compounds of formula (Ia), (IIa) and preferred embodiments thereof are produced by
(i) polymerization of a monomer (3) obtained by a cycloaddition of an azide and an alkyne and cyclization of the polymer obtained to obtain a cyclic polymer core A; and
(ii) (ii) reacting the cyclic polymer core A obtained in step (i) with groups L-X or X, wherein L and X are as defined earlier; and
(iii) if compounds of formula (IIa) are produced, reacting the reaction product of step (ii) with a paramagnetic metal ion, preferably in the form of its salt.

The cyclic polymer core A obtained in step (i) suitably comprises 3 or 4 reactive groups R″ or precursors thereof which react with in a subsequent step (ii) with the group L-X or X, if L is already a part of the cyclic polymer core obtained in step (i), as described on the previous page.

L-X or X preferably comprise a functional group which can react with the R″ groups of A. If R″ is a precursor of a reactive group, said precursor may nee d to be activated, e.g. deprotected, to form a reactive group, e.g. a free amine or an activated carboxylic acid which will then react with L-X or X. R″ is either chemically inert to the conditions in step (i) or it has to be protected, i.e. transformed into a precursor of a reactive group and then activated after step (i) is finished to react with L-X or X. An example of such a precursor of R″ is a nitro group—as shown on the previous page—which can be turned into a reactive group R″, i.e. a free amine, by reducing said nitro group. Other examples are benzylamines, azido groups or ester groups.

As mentioned above, the L moiety of L-X or X may comprise a functional group and examples of such functional groups include hydroxy, amino, sulfhydryl, carbonyl (including aldehyde and ketone), carboxylic acid and thiophosphate groups. With regard to X, some other functional groups may need to be protected, e.g. carboxylic groups and these groups need to be deprotected, preferably after the attachment of X.

Reactive groups R″ are preferably selected from succinimidyl ester, sulpho-succinimidyl ester, 4-sulfo-2,3,5,6-tetrafluorophenol (STP) ester, isothiocyanate, maleimide, haloacetamide, acid halide, hydrazide, vinylsulphone, dichlorotriazine and phosphoramidite. More preferred the reactive group R″ is a succinimidyl ester of a carboxylic acid, an isothiocyanate, a maleimide, a haloacetamide or a phosphoramidite.

Generally, to obtain compounds of formula (II) and preferred embodiments thereof, X can be transformed into X′ by complex formation with a suitable paramagnetic metal ion M, preferably in the form of its salt (e.g. like Gd(III) acetate or Gd(III) Cl₃).

The invention is illustrated by the examples in the corresponding section of this patent application.

The compounds of formula (II) and preferred embodiments thereof may be used as MR contrast agents. For this purpose, the compounds of formula (II) are formulated with conventional physiologically tolerable carriers like aqueous carriers, e.g. water and buffer solution and optionally excipients.

Hence in a further aspect the present invention provides a composition comprising a compound of formula (II) and at least one physiologically tolerable carrier.

In a further aspect the invention provides a composition comprising a compound of formula (II) and at least one physiologically tolerable carrier for use as MR imaging contrast agent or MR spectroscopy contrast agent.

To be used as contrast agents for MR imaging or spectroscopy of the human or non-human animal body, said compositions need to be suitable for administration to said body. Suitably, the compounds of formula (II) and optionally pharmaceutically acceptable excipients and additives may be suspended or dissolved in at least one physiologically tolerable carrier, e.g. water or buffer solutions. Suitable additives include for example physiologically compatible buffers like tromethamine hydrochloride, chelators such as DTPA, DTPA-BMA or compounds of formula (I) or preferred embodiments thereof, weak complexes of physiologically tolerable ions such as calcium chelates, e.g. calcium DTPA, CaNaDTPA-BMA, compounds of formula (I) or preferred embodiments thereof wherein X forms a complex with Ca²⁺ or CaNa salts of compounds of formula (I) or preferred embodiments thereof, calcium or sodium salts like calcium chloride, calcium ascorbate, calcium gluconate or calcium lactate. Excipients and additives are further described in e.g. WO-A-90/03804, EP-A-463644, EP-A-258616 and U.S. Pat. No. 5,876,695, the content of which are incorporated herein by reference.

Another aspect of the invention is the use of a composition comprising a compound of formula (II) and at least one physiologically tolerable carrier as MR imaging contrast agent or MR spectroscopy contrast agent.

Yet another aspect of the invention is a method of MR imaging and/or MR spectroscopy wherein a composition comprising a compound of formula (II) and at least one physiologically tolerable carrier is administered to a subject and the subject is subjected to an MR procedure wherein MR signals are detected from the subject or parts of the subject into which the composition distributes and optionally MR images and/or MR spectra are generated from the detected signals.

In a preferred embodiment, the subject is a living human or non-human animal body.

In a further preferred embodiment, the composition is administered in an amount which is contrast-enhancing effective, i.e. an amount which is suitable to enhance the contrast in the MR procedure.

In a preferred embodiment, the subject is a living human or non-human animal being and the method of MR imaging and/or MR spectroscopy is a method of MR angiography, more preferred a method of MR peripheral angiography, renal angiography, supra aortic angiography, intercranial angiography or pulmonary angiography.

In another preferred embodiment, the subject is a living human nor non-human animal being and the method of MR imaging and/or MR spectroscopy is a method of MR tumour detection or a method of tumour delineation imaging.

In another aspect, the invention provides a method of MR imaging and/or MR spectroscopy wherein a subject which had been previously administered with a composition comprising a compound of formula (II) and at least one physiologically tolerable carrier is subjected to an MR procedure wherein MR signals are detected from the subject or parts of the subject into which the composition distributes and optionally MR images and/or MR spectra are generated from the detected signals.

The term “previously been administered” means that any step requiring a medically-qualified person to administer the composition to the patient has already been carried out before the method of MR imaging and/or MR spectroscopy according to the invention is commenced.

### EXAMPLES

### Example 1

### Preparation of a Compound of Formula (II) Comprising a Cyclic Polymer Core A of Formula (VI)

### Example 1a

### Preparation of a Cyclic Polymer Core A of Formula (VI) Comprising a Moiety L-T

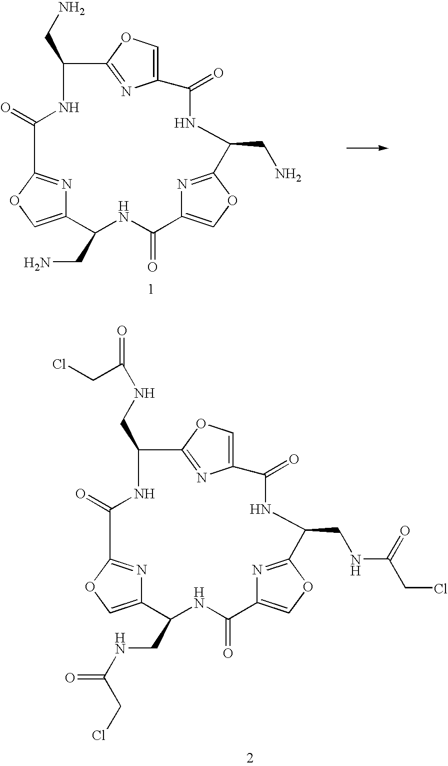

Compound 1 is prepared according to D. Mink, et al., Tetrahedron Lett. 1998, 39, 5709-5712.

Compound 1 (1.0 g, 2.18 mmol) is dissolved in acetonitrile (50 mL) and chloroacetyl chloride (0.69 mL, 8.7 mmol) is added followed by triethylamine (0.9 mL, 6.5 mmol). After 1 h the reaction mixture is crashed into water (500 mL) and the precipitate is filtered off to give compound 2.

### Example 1b

### Reaction of the Compound of Example 1a) with a Protected Chelator X

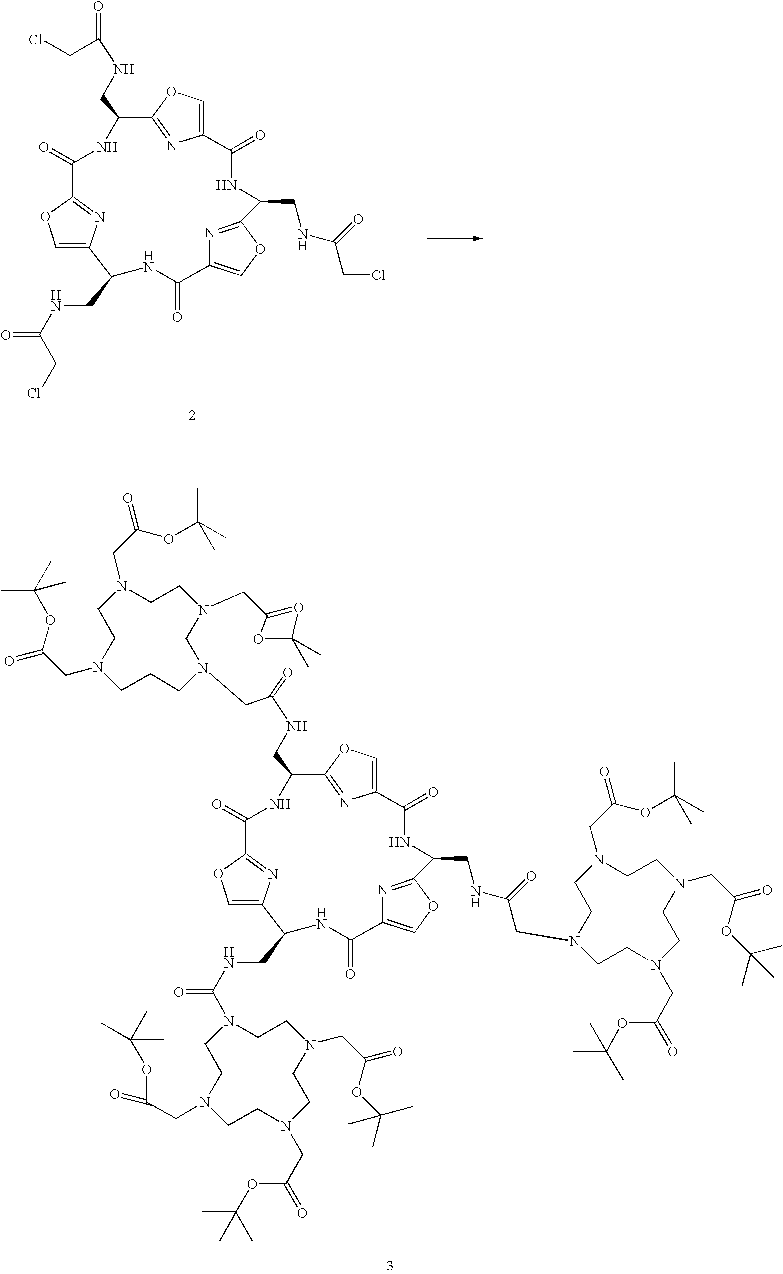

Compound 2 (1.5 g, 2.18 mmol) is dissolved in acetonitrile and 1,4,7,10-tetraazacyclododecane-1,4,7-triacetic acid tri-t-butyl ester hydrobromide (5.2 g, 8.8 mmol) is added followed by triethylamine (2.4 mL, 17.6 mmol). After 24 h the reaction mixture is concentrated to give compound 3 in a crude reaction mixture which is used in the next step without purification.

### Example 1c

### Deprotection of the Chelator X

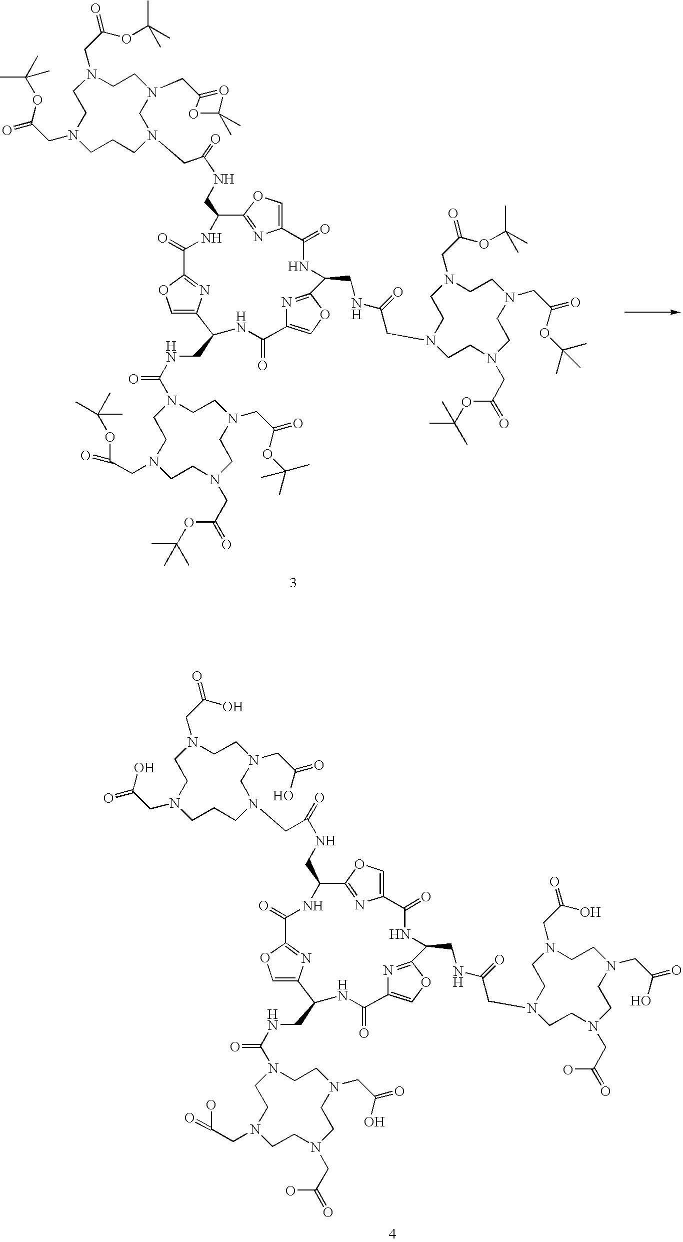

The reaction mixture containing the crude compound 3 is dissolved in formic acid (50 mL) and refluxed for 12 h and then concentrated to give compound 4 in a crude reaction mixture that is used in the next step without purification.

### Example 1d

### Reaction of the Compound of Example 1c) with Gd³⁺ to Form X′

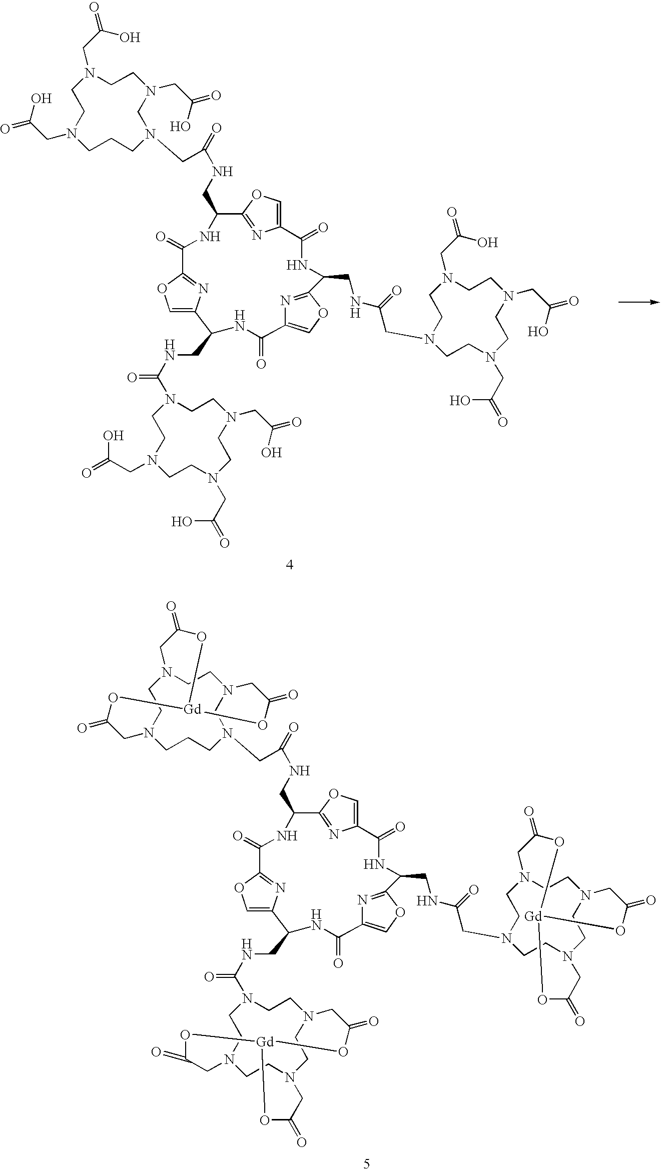

The reaction mixture containing the crude compound 4 is dissolved in water (50 mL) and Gd(OAc)₃ (2.9 g, 8.8 mmol) is added. The reaction mixture is stirred for 24 h and then concentrated. The crude reaction mixture is purified by HPLC to give compound 5.

### Example 2

### Preparation of a Compound of Formula (IIc)

### Example 2a

### Preparation of the Azide

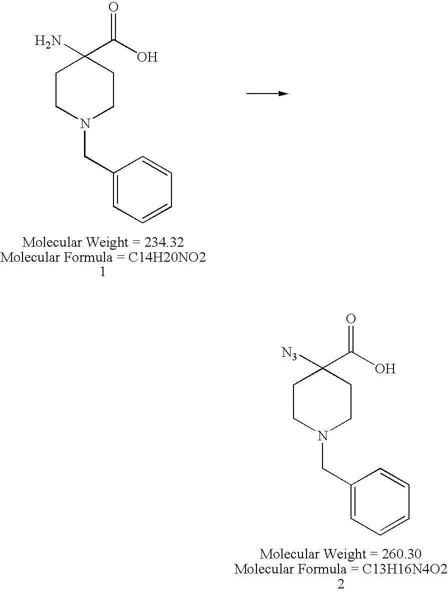

Compound 1 (0.5 g, 2.1 mmol) which is synthesized according to Journal of Medicinal Chemistry 45(18), 2002, 3972-3983 is dissolved in a methanol:water mixture (2:1, 30 mL) and K₂CO₃ (0.58 g, 4.2 mmol) is added followed by CuSO₄×5H₂O (7 mg, 0.028 mmol). To the stirred mixture is added a TfN₃ solution in dichloromethane (2 mL, 2 M) according to Organic Letters 3(5), 2001, 781-783. After 18 h the organic solvents are removed and the aqueous solution is diluted with water (50 mL) and acidified to pH 6 using concentrated HCl. The aqueous phase is washed with ethyl acetate (50 mL) and then acidified to pH 2 using concentrated HCl. The product is removed from the aqueous phase by extraction with ethyl acetate (50 mL). The organic phase is dried and evaporated to give compound 2.

### Example 2b

### Cycloaddition of the Azide with an Alkyne

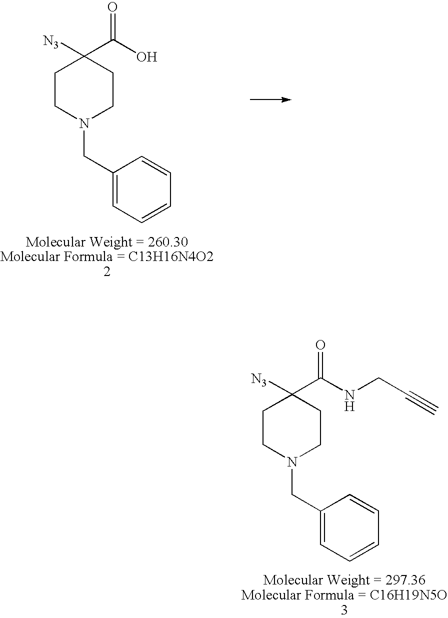

Compound 2 (1.0 g, 3.8 mmol) is dissolved in THF (10 mL), and 1,1-carbonyldiimidazole (0.7 g, 4.2 mmol) is added. The solution is refluxed for 5 h and then propargylamine (0.4 mL, 5.7 mmol) is added. After additional 5 h, the reaction is crashed into an acidified aqueous solution (25 mL, 0.5 M HCl) and the formed precipitate is filtered off to give compound 3.

### Example 2c

### Polymerization/Cyclization of the Monomer

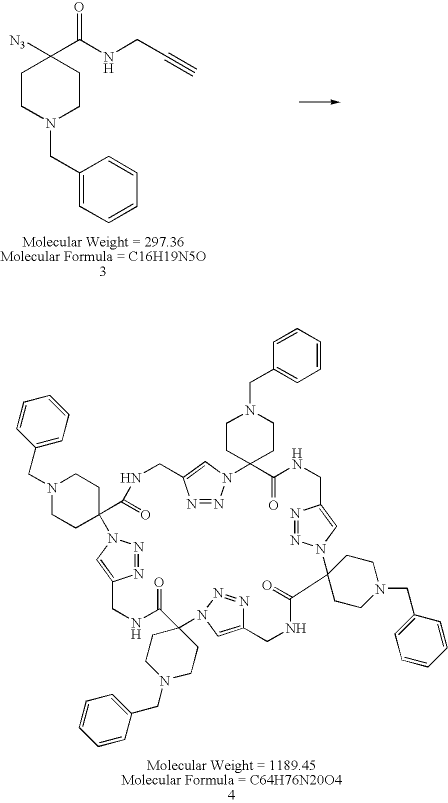

Compound 3 (1.0 g, 3.4 mmol) is dissolved in a THF:water mixture (9:1, 10 mL) and then ascorbic acid (1.0 g, 5.7 mmol), NaOAc (0.7 g, 8.5 mmol) and CuSO₄×5H₂O (0.1 g, 0.4 mmol) is added. The stirred reaction mixture is refluxed for 5 h and then crashed into water (10 mL). The precipitate is filtered off to give compound 4.

### Example 2d

### Generation of a Reactive Group for Attachment of the Chelator X

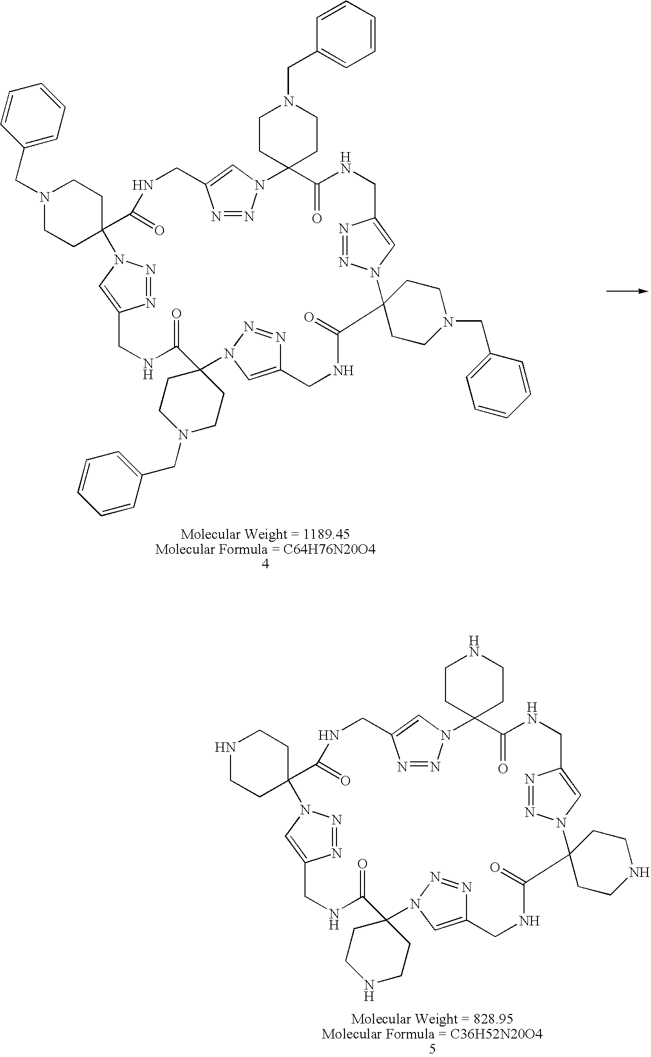

To compound 4 (10 g, 8.4 mmol) dissolved in EtOH (100 mL) is added Pd(OH)₂/C (2 g, 20%) followed by addition of ammonium formate (1.1 g 16.8 mmol). The mixture is refluxed for 18 h and then filtered and concentrated to give compound 5.

### Example 2e

### Attachment of a Protected Chelator X

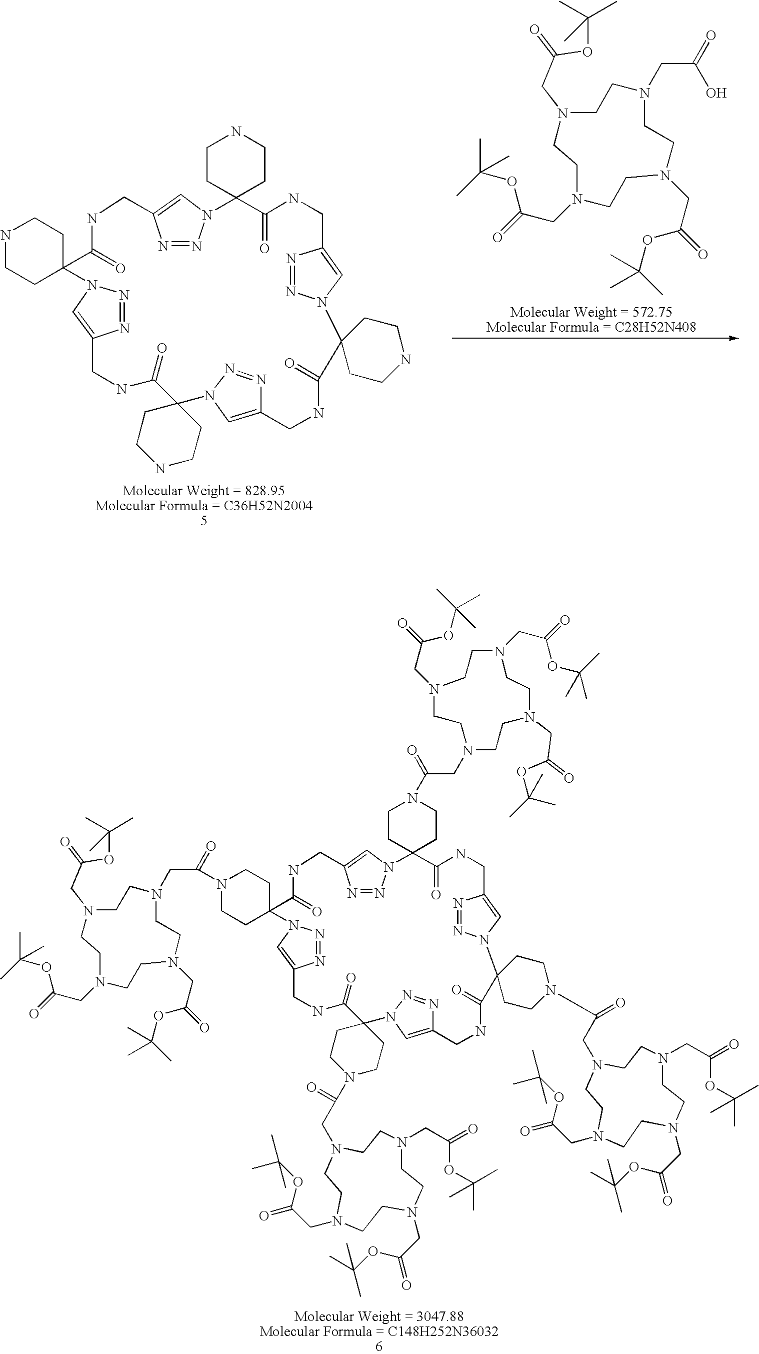

1,4,7,10-tetraazacyclododecane-1,4,7,10-tetraacetic acid tris-tertbutyl ester (1.0 g, 1.7 mmol) is dissolved in DMF (5 mL). HATU (0.66 g, 1.7 mmol) is added followed by N,N-diisopropylethylamine (0.6 mL, 3.4 mmol) Compound 5 (0.36 g, 0.43 mmol) is added and after a 18 h reaction the reaction mixture is crashed into water (100 mL) and the precipitate is filtered off to give compound 6.

### Example 2f

### Deprotection of the Chelator X

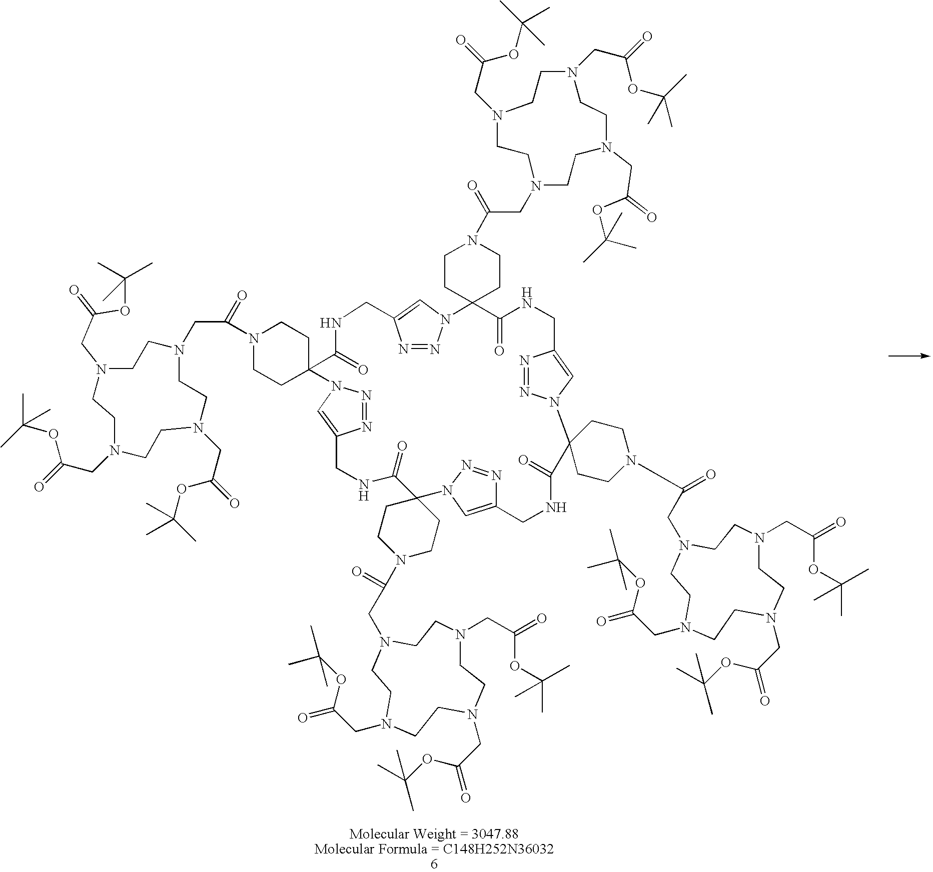

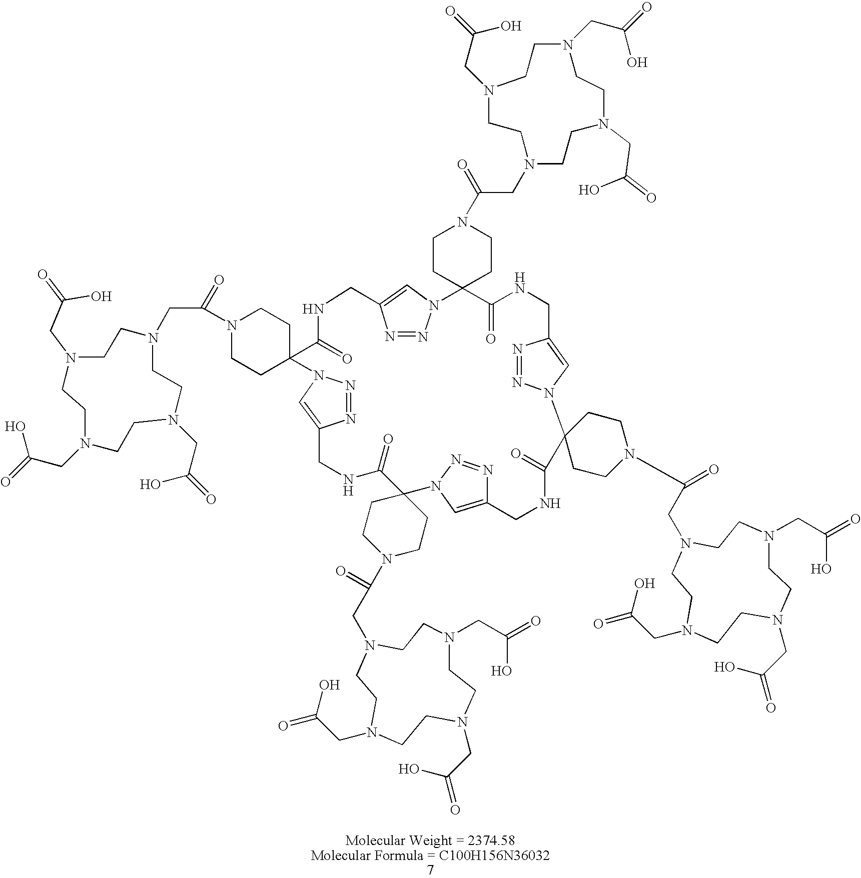

Compound 6 is dissolved in formic acid (50 mL) and refluxed for 12 h and then concentrated to give compound 7 as a crude reaction mixture that is used in the next step without purification.

### Example 2g

### Reaction of the Compound of Example 2f) with Gd³⁺ to Form X′

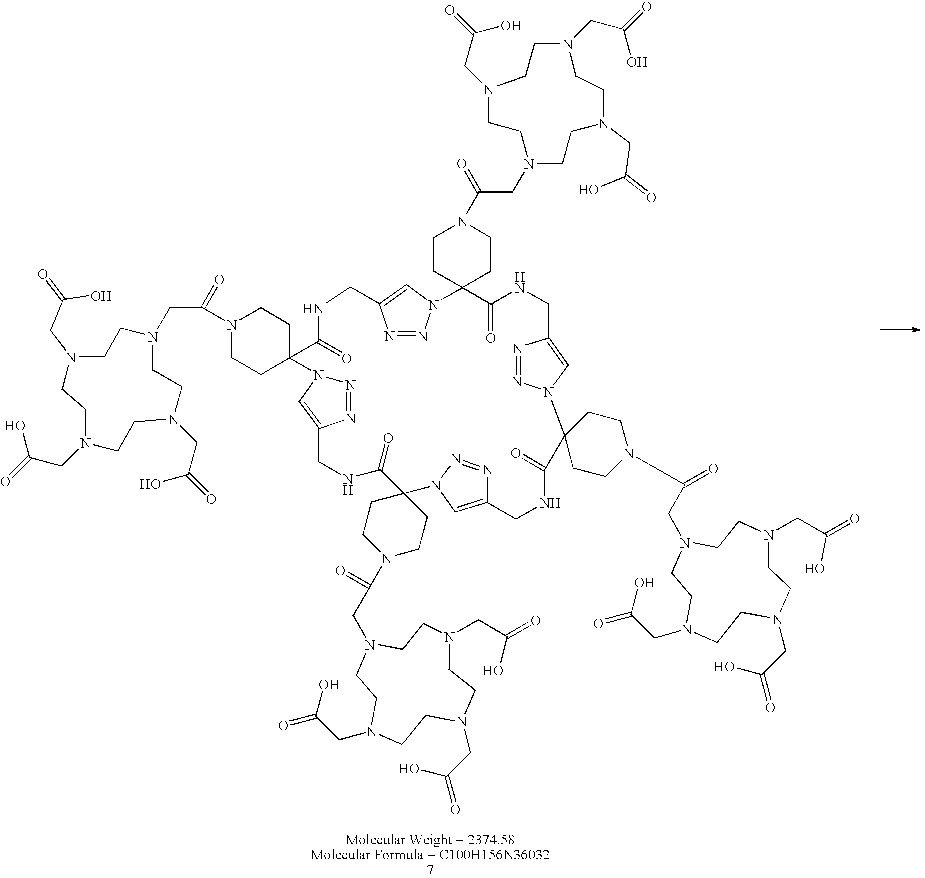

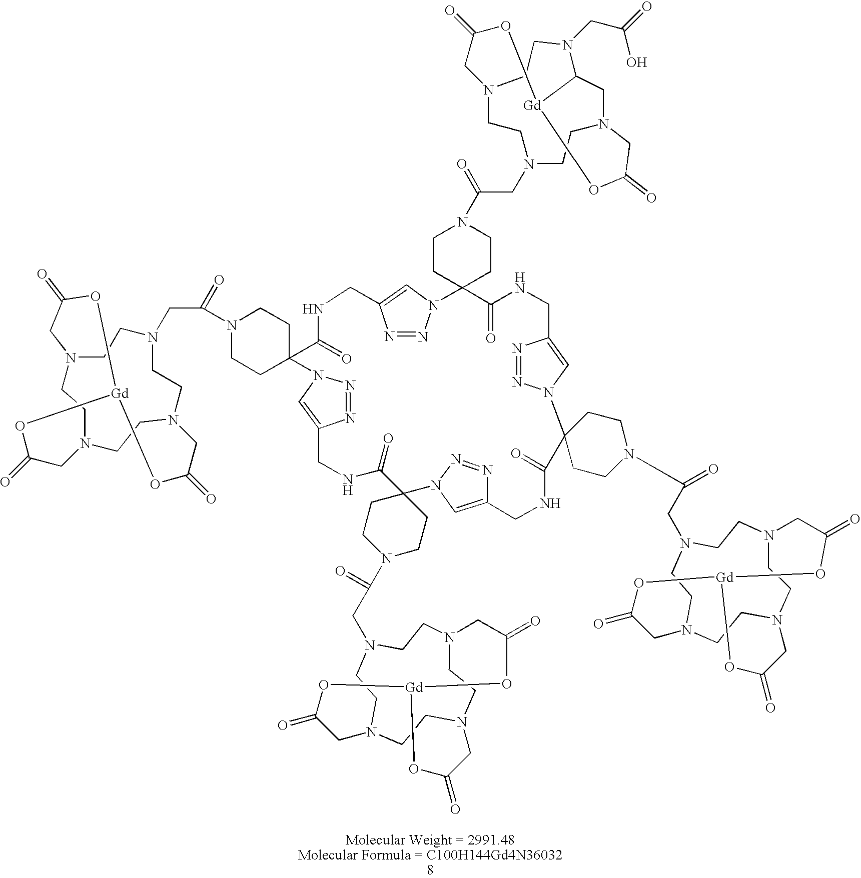

The crude compound 7 is dissolved in water (50 mL) and Gd(OAc)₃ (2.9 g, 8.8 mmol) is added. The reaction mixture is stirred for 24 h and then concentrated. The crude reaction mixture is purified by HPLC to give compound 8.

1. Compound of formula (II) consisting of a cyclic polymer core A and groups -L-X′ attached to said core
A-(L-X′)n (II)
wherein
A denotes a cyclic polymer which is comprised of 3 or 4 identical monomers which are connected by amide bonds;
L may be present or not and if present is that same or different and denotes a linker moiety,
X′ is the same or different and denotes a paramagnetic chelate consisting of a chelator X and a paramagnetic metal ion M; and
n denotes an integer of 3 or 4.

2. Compound according to claim 1 wherein A is comprised of 3 or 4 identical monomers and each of said monomers comprises a 1,2,3-triazole of formula (IIIa)

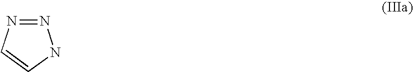

3. Compound according to claim 1 wherein A is a cyclic polymer of formula (IV) 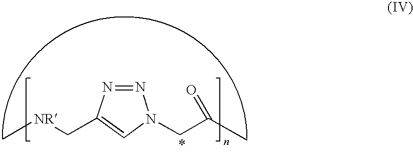
wherein
R′ denotes a group to improve solubility;
* denotes the attachment of the A to L-X′
n is defined as in claim 1 and is preferably 4.

4. Compound according to claim 1 wherein A is a cyclic polymer of formula (V) 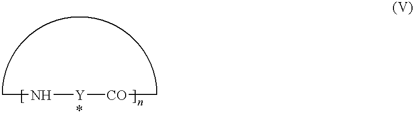
wherein
n is defined as in claim 1 and is preferably 3;
Y denotes a moiety CR1R2-CO-heterocycle or CR1R2-heterocycle, wherein both R1 and R2 are present and are the same or different and denote R′ as defined as a group to improve solubility or only R1 or R2 is present and denotes R′;
* denotes the attachment of the A to L-X′

5. Compound according to claim 1 wherein A is a cyclic polymer of formula (VI) 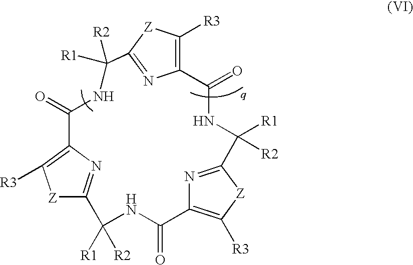
wherein
z denotes O, S or NR4;
denotes R′ as defined as a group to improve solubility;
R1 and R2 are present and are the same or different; and
q is an integer of 1 or 2.

6. Compound according to claim 1 wherein A is a cyclic polymer of formula (VII) 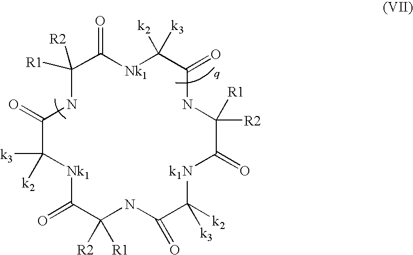
wherein
R1, R2 are present and are the same or different;
q is an integer of 1 or 2;
k₁ denotes H or CH₃ and k₁ and either of k₂ or k₃ form a saturated or non-saturated nitrogen heterocycle, preferably a 5- or 6-membered nitrogen heterocycle and most preferably pyrrolidine.

7. Compounds according to claim 1 wherein L is present.

8. Compounds according to claim 1 wherein L is —CZ¹Z²-CO—N(Z³)-*
wherein
* denotes the attachment of the core A to said linker moiety;
Z¹ and Z² independently of each other denote a hydrogen atom, a hydroxyl group or a C₁-C₈-alkyl group optionally substituted by hydroxyl, amino or mercapto groups, and/or optionally comprising an oxo-group; and
Z³ stands for H, C₁-C₈-alkyl, optionally substituted with one or more hydroxyl or amino groups.

9. Compounds according to claim 1 wherein L comprises benzene or N-heterocycles and the core A is attached to either one of the nitrogen atoms in said N-heterocycles or to a carbon atom in said N-heterocycles or in benzene.

10. Compounds according to claim 1 wherein X is a cyclic chelator of formula (VIII) 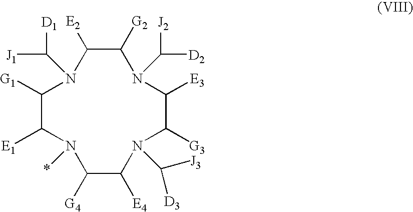
wherein
* denotes the attachment of L, if present, or the core A, if L is not present;
E₁ to E₄ independent of each other is selected from H, CH₂, CH₃, OCH₃, CH₂OH, CH₂OCH₃, OCH₂CH₃, OCH₂CH₂OH, COOH, COOCH₃, COOCH₂CH₃, C(O)NH₂, C(O)N(CH₃)₂, C(O)N(CH₂CH₃)CH₃ or C(O)N(CH₂CH₃)₂;
G₁ to G₄ independent of each other is selected from H, CH₂, CH₃, OCH₃, CH₂OH, CH₂OCH₃, OCH₂CH₃, OCH₂CH₂OH, COOH, COOCH₃, COOCH₂CH₃, C(O)NH₂, C(O)N(CH₃)₂, C(O)N(CH₂CH₃)CH₃, or C(O)N(CH₂CH₃)₂;
D₁ to D₃ independent of each other is selected from H, OH, CH₃, CH₂CH₃, CH₂OH, CH₂OCH₃, OCH₂CH₃, OCH₂CH₂OH or OCH₂C₆H₅; and
J₁ to J₃ independent of each other is selected from COOH, P(O)(OH)₂, P(O)(OH)CH₃, P(O)(OH)CH₂CH₃, P(O)(OH)(CH₂)₃CH₃, P(O)(OH)Ph, P(O)(OH)CH₂Ph, P(O)(OH)OCH₂CH₃, CH(OH)CH₃, CH(OH)CH₂OH, C(O)NH₂, C(O)NHCH₃, C(O)NH(CH₂)₂CH₃, OH or H.

11. Compound according to claim 1 wherein X is a residue selected from DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

12. (canceled)

13. Compound according to claim 1 wherein all L and/or all X′ are the same.

14. Composition comprising the compound according to claim 1 and at least one physiologically tolerable carrier.

15. Composition according to claim 14 for use as MR imaging contrast agent or MR spectroscopy contrast agent.

16. Use of the composition of claim 14 as MR imaging contrast agent or MR spectroscopy contrast agent.

17. Method of MR imaging and/or MR spectroscopy wherein the composition of claim 15 is administered to a subject and the subject is subjected to an MR procedure wherein MR signals are detected from the subject or parts of the subject into which the composition distributes and optionally MR images and/or MR spectra are generated from said detected signals.

18. (canceled)

19. Process for the preparation of compounds according to claim 2 by
(i) polymerization of a monomer (3) 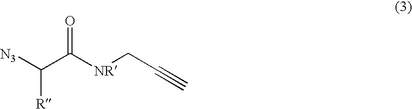
obtained by a cycloaddition of an azide and an alkyne and cyclization of the polymer obtained to obtain a cyclic polymer core A; and
(ii) (ii) reacting the cyclic polymer core A obtained in step (i) with groups L-X or X, wherein L may be present or not and if present is that same or different and denotes a linker moiety and X is a chelator; and
(iii) reacting the reaction product of step (ii) with a paramagnetic metal ion, preferably in the form of its salt.

20. Process for the preparation of compounds according to claim 4 by
(i) polymerization and cyclization of monomers H₂N—CR1R2-heterocycle-COOH or H₂N—CR1R2-CO-heterocycle-COOH, wherein R1 and R2 are as defined in claim 4;
(ii) reacting the cyclic polymer core A obtained in step (i) with groups L-X or X, wherein L may be present or not and if present is that same or different and denotes a linker moiety and X is a chelator; and
(iii) reacting the reaction product of step (ii) with a paramagnetic metal ion, preferably in the form of its salt.

21. (canceled)
